(19) United States
(12) Patent Application Publication
(10) Pub. No.: US 2019/0046103 A1
(43) Pub. Date: Feb. 14, 2019

(54) Flow Measuring Assembly

(71) Applicants: Jasmine Henderson, Atlantic Beach, FL (US); David Doward, Atlantic Beach, FL (US)

(72) Inventors: Jasmine Henderson, Atlantic Beach, FL (US); David Doward, Atlantic Beach, FL (US)

(21) Appl. No.: 15/673,006

(22) Filed: Aug. 9, 2017

(51) Int. Cl. A61B 5/00; G01F 1/74; G01F 15/06; A61J 13/00

(52) U.S. Cl. CPC A61B 5/4288; A61B 5/6813; A61J 13/00; G01F 15/063; G01F 1/74

(57) ABSTRACT

A flow measuring assembly for tracking a fluid volume of breast milk consumed by an infant includes a shield that is selectively worn on a breast. The shield has a nipple thereon and the nipple on the shield is aligned with a nipple on the breast. The nipple on the shield is perforated to pass breast milk therethrough. A measuring unit is coupled to the shield. The measuring unit is in fluid communication with the nipple on the shield to measure a flow of the breast milk through the nipple on the shield. The measuring unit is in electrical communication with an electronic device. In this way the flow of breast milk by volume is communicated to the electronic device for tracking the flow of breast milk by volume.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to measuring devices and more particularly pertains to a new measuring device for tracking a fluid volume of breast milk consumed by an infant.

### BRIEF SUMMARY OF THE INVENTION

An embodiment of the disclosure meets the needs presented above by generally comprising a shield that is selectively worn on a breast. The shield has a nipple thereon and the nipple on the shield is aligned with a nipple on the breast. The nipple on the shield is perforated to pass breast milk therethrough. A measuring unit is coupled to the shield. The measuring unit is in fluid communication with the nipple on the shield to measure a flow of the breast milk through the nipple on the shield. The measuring unit is in electrical communication with an electronic device. In this way the flow of breast milk by volume is communicated to the electronic device for tracking the flow of breast milk by volume.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a flow measuring assembly according to an embodiment of the disclosure.

FIG. 2 is a left side view of an embodiment of the disclosure.

FIG. 3 is a back view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

FIG. 6 is a schematic view of an embodiment of the disclosure.

### DETAILED DESCRIPTION OF THE INVENTION

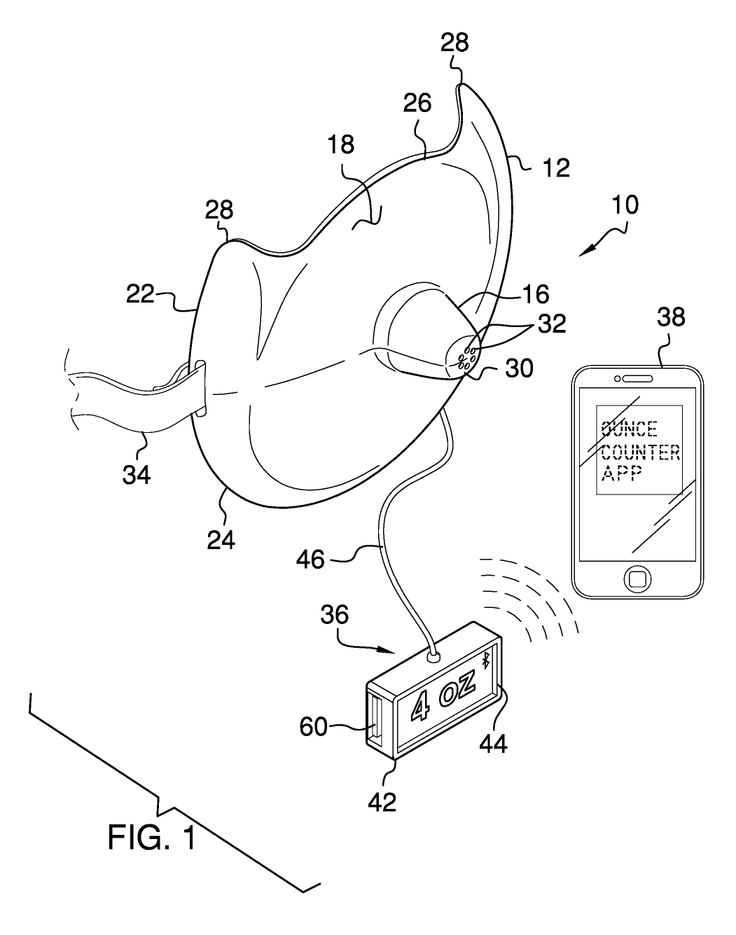

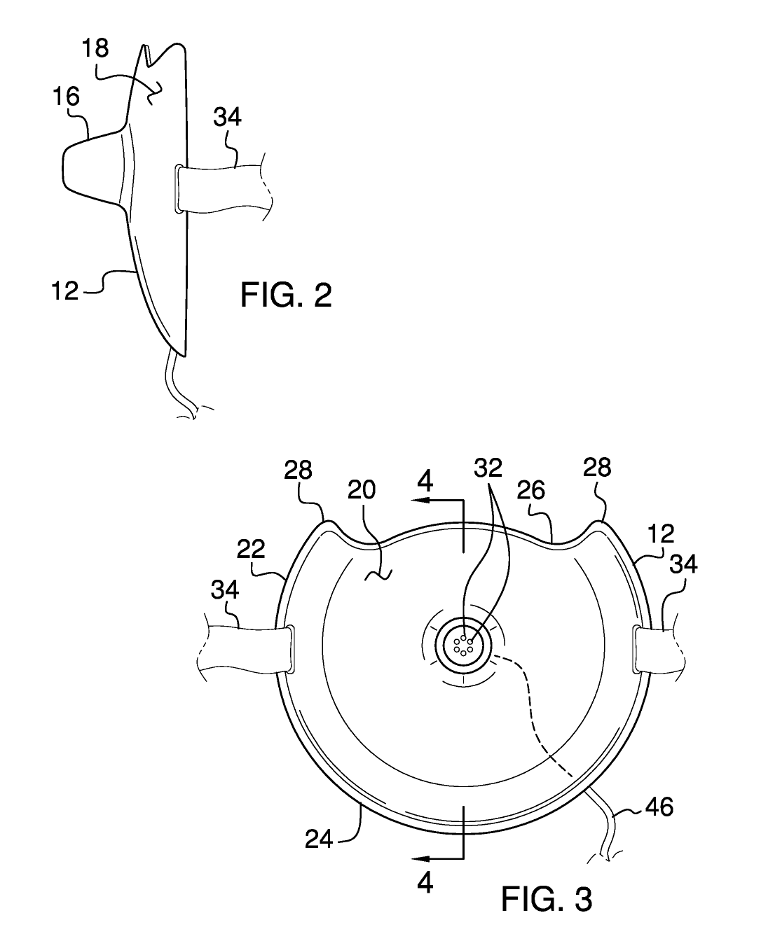

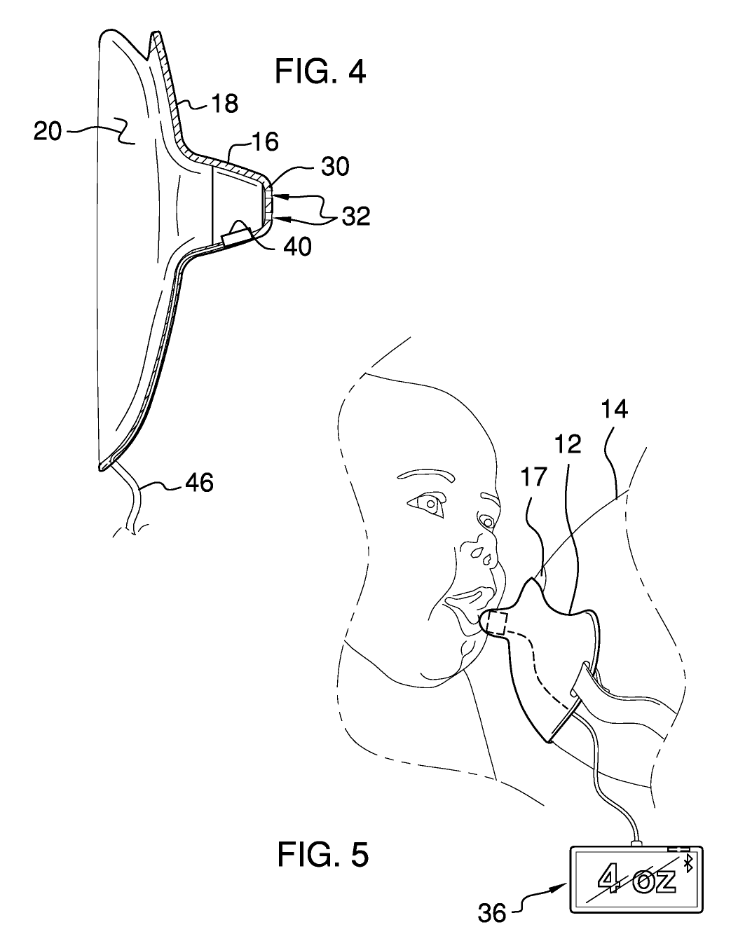

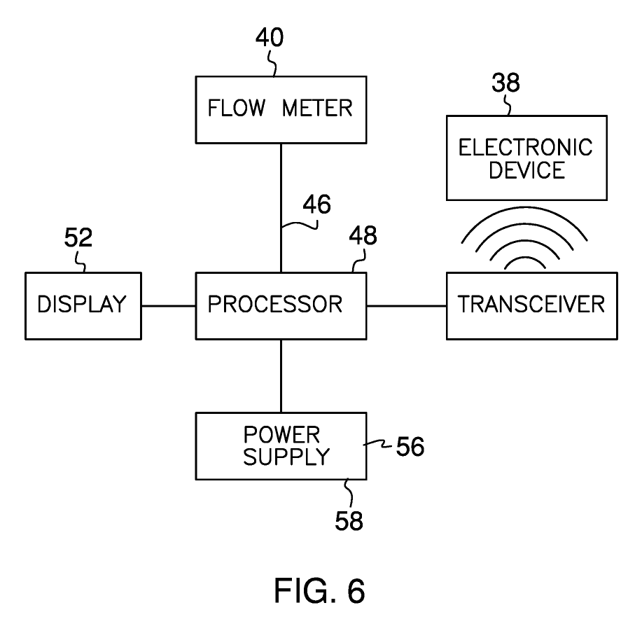

With reference now to the drawings, and in particular to FIGS. 1 through 6 thereof, a new measuring device embodying the principles and concepts of an embodiment of the disclosure and generally designated by the reference numeral 10 will be described.

As best illustrated in FIGS. 1 through 6, the flow measuring assembly 10 generally comprises a shield 12 that may be worn on a female breast 14. The shield 12 is worn when the female is breast feeding an infant. The shield 12 has a nipple 16 thereon and the nipple 16 on the shield 12 is aligned with a nipple 17 on the breast 14 when the shield 12 is worn on the breast 14. The nipple 16 on the shield 12 is perforated to pass breast milk therethrough. Moreover, the shield 12 is comprised of a deformable material such as silicone or the like to enhance comfort while wearing the shield 12.

The shield 12 has a first surface 18, a second surface 20 and a peripheral edge 22 extending therebetween. Each of the first 18 and second 20 surfaces is concavely arcuate between the peripheral edge 22 to conform to a curve of the breast 14. The peripheral edge 22 has a bottom side 24 and a top side 26. The bottom side 24 is concavely arcuate with respect to the top side 26 to conform the curve of the breast 14. The bottom side 24 has a pair of intersections 28 with the top side 26 and the top side 26 undulates between each of the intersections 28.

The first 18 and second 20 surfaces extend forwardly from the shield 12 to define the nipple 16. Moreover, the nipple 16 is centrally positioned on the shield 12. The nipple 16 has a distal end 30 with respect to the shield 12. The distal end 30 has a plurality of openings 32 extending therethrough to pass the breast milk therethrough. A pair of straps 34 is each coupled to the peripheral edge 22 of the shield 12. Each of the straps 34 is matable to each other and each of the straps is selectively wrapped around the user. In this way the shield 12 is retained on the breast 14.

A measuring unit 36 is coupled to the shield 12 and the measuring unit 36 is in fluid communication with the nipple 16 on the shield 12. The measuring unit 36 measures a flow of the breast milk through the nipple 16 on the shield 12. In this way a mother may track how much milk the infant has consumed. The measuring unit 36 is in electrical communication with an electronic device 38 such as a smart phone or the like. The flow of breast milk by volume may be communicated to the electronic device 38 for tracking the flow of breast milk by volume.

The measuring unit 36 comprises a flow meter 40 that is coupled to the first surface 18 of the shield 12. The flow meter 40 is positioned in the nipple 16. Thus, the flow meter 40 is in fluid communication with the breast milk flowing through the nipple 16 on the shield 12. The flow meter 40 may be an electronic fluid flow meter 40 or the like.

A housing 42 is provided that has a first wall 44. A conductor 46 is coupled between the housing 42 and the shield 12 and the conductor 46 is electrically coupled to the flow meter 40. A processor 48 is positioned within the housing 42 and the processor 48 is electrically coupled to the conductor 46. In this way the processor 48 is in electrical communication with the flow meter 40. The conductor 46 may be an insulated wire or the like.

A transceiver 50 is positioned within the housing 42 and the transceiver 50 is electrically coupled to the processor 48. The transceiver 50 is in wireless electrical communication with the electronic device 38. The transceiver 50 may be a radio frequency transceiver 50 or the like. Moreover, the transceiver 50 may employ a WPAN signal and Bluetooth communication protocols. The electronic device 38 may store a program or app for measuring the fluid volume.

A display 52 is coupled to the first wall 44 of the housing 42 and the display 52 displays indicia 54. The indicia 54 comprise numbers corresponding to a volume in fluid ounces with respect to the flow of breast milk. The display 52 is electrically coupled to the processor 48 and the display 52 may be an LED display or the like.

A power supply 56 is removably positioned within the housing 42 and the power supply 56 is electrically coupled to the processor 48. The power supply 56 comprises at least one battery 58. A battery cover 60 is removably coupled to the housing 42 and the power supply 56 is positioned beneath the battery cover 60.

In use, the shield 12 is worn on the breast 14 such that the nipple 16 on the breast 14 is aligned with the nipple 16 on the shield 12. The transceiver 50 is selectively synched with the electronic device 38 to track the flow of breast milk flowing through the nipple 16 on the shield 12. The infant suckles on the nipple 16 on the shield 12 to consume the breast milk. The flow meter 40 measures the flow of breast milk through the nipple 16 on the shield 12. The display 52 on the housing 42 displays the fluid volume of breast milk that has been consumed. Thus, the mother can track how much breast milk the infant has consumed. In this way the mother can follow a doctor's advice with respect to feeding the infant. Additionally, the fluid volume of the breast milk may be viewed on the electronic device 38.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A flow measuring assembly being configured to be worn on a breast thereby facilitating a flow of breast milk to be measured by volume, said assembly comprising:
a shield being configured to be worn on a breast, said shield having a nipple thereon wherein said nipple on said shield is configured to be aligned with a nipple on the breast, said nipple on said shield being perforated wherein said nipple on said shield is configured to pass breast milk therethrough; and
a measuring unit being coupled to said shield, said measuring unit being in fluid communication with said nipple on said shield wherein said measuring unit is configure to measure a flow of the breast milk through said nipple on said shield, said measuring unit being configured to be in electrical communication with an electronic device thereby facilitating the flow of breast milk by volume to be communicated to the electronic device for tracking the flow of breast milk by volume.

2. The assembly according to claim 1, wherein said shield has a first surface, a second surface and a peripheral edge extending therebetween, each of said first and second surface being concavely arcuate between said peripheral edge wherein said shield is configured to conform to a curve of the breast, said peripheral edge having a bottom side and a top side.

3. The assembly according to claim 2, wherein said bottom side is concavely arcuate with respect to said top side, said bottom side having a pair of intersections with said top side, said top side undulating between each of said intersections.

4. The assembly according to claim 2, wherein said first and second surfaces extend forwardly from said shield to define said nipple, said nipple having a distal end with respect to said shield, said distal end having a plurality of openings extending therethrough wherein each of said openings is configured to pass the breast milk therethrough.

5. The assembly according to claim 2, wherein said measuring unit comprises a flow meter being coupled to said first surface of said shield, said flow meter being positioned in said nipple wherein said flow meter is configured to be in fluid communication with the breast milk flowing through said nipple on said shield.

6. The assembly according to claim 5, further comprising:
a housing having a first wall; and
a conductor being coupled between said housing and said shield, said conductor being electrically coupled to said flow meter.

7. The assembly according to claim 6, further comprising a processor being positioned within said housing, said processor being electrically coupled to said conductor such that said processor is in electrical communication with said flow meter.

8. The assembly according to claim 7, further comprising a transceiver being positioned within said housing, said transceiver being electrically coupled to said processor, said transceiver being configured to be in wireless electrical communication with the electronic device.

9. The assembly according to claim 7, further comprising a display being coupled to said first wall of said housing, said display being configured to display indicia, said indicia comprising numbers corresponding to a volume in fluid ounces with respect to the flow of breast milk, said display being electrically coupled to said processor.

10. The assembly according to claim 7, further comprising a power supply being removably positioned within said housing, said power supply being electrically coupled to said processor, said power supply comprising at least one battery.

11. A flow measuring assembly being configured to be worn on a breast thereby facilitating a flow of breast milk to be measured by volume, said assembly comprising:
a shield being configured to be worn on a breast, said shield having a nipple thereon wherein said nipple on said shield is configured to be aligned with a nipple on the breast, said nipple on said shield being perforated wherein said nipple on said shield is configured to pass breast milk therethrough, said shield having a first surface, a second surface and a peripheral edge extending therebetween, each of said first and second surface being concavely arcuate between said peripheral edge wherein said shield is configured to conform to a curve of the breast, said peripheral edge having a bottom side and a top side, said bottom side being concavely arcuate with respect to said top side, said bottom side having a pair of intersections with said top side, said top side undulating between each of said intersections, said first and second surfaces extending forwardly from said shield to define said nipple, said nipple having a distal end with respect to said shield, said distal end having a plurality of openings extending therethrough wherein each of said openings is configured to pass the breast milk therethrough;
a measuring unit being coupled to said shield, said measuring unit being in fluid communication with said nipple on said shield wherein said measuring unit is configure to measure a flow of the breast milk through said nipple on said shield, said measuring unit being configured to be in electrical communication with an electronic device thereby facilitating the flow of breast milk by volume to be communicated to the electronic device for tracking the flow of breast milk by volume, said measuring unit comprising:
a flow meter being coupled to said first surface of said shield, said flow meter being positioned in said nipple wherein said flow meter is configured to be in fluid communication with the breast milk flowing through said nipple on said shield,
a housing having a first wall,
a conductor being coupled between said housing and said shield, said conductor being electrically coupled to said flow meter,
a processor being positioned within said housing, said processor being electrically coupled to said conductor such that said processor is in electrical communication with said flow meter,
a transceiver being positioned within said housing, said transceiver being electrically coupled to said processor, said transceiver being configured to be in wireless electrical communication with the electronic device,
a display being coupled to said first wall of said housing, said display being configured to display indicia, said indicia comprising numbers corresponding to a volume in fluid ounces with respect to the flow of breast milk, said display being electrically coupled to said processor, and
a power supply being removably positioned within said housing, said power supply being electrically coupled to said processor, said power supply comprising at least one battery.
